(19) United States
(12) Patent Application Publication
(10) Pub. No.: US 2010/0209491 A1
(43) Pub. Date: Aug. 19, 2010

(54) METHOD FOR ENHANCING SERUM STABILITY AND LOWERING IMMUNE RESPONSE OF SIRNA DOWN-REGULATING GENE EXPRESSION OF HBV OR HCV

(75) Inventors: Soo In Kim, Yongin-si (KR); Duckhyang Shin, Yongin-si (KR); Hyeon Lee, Yongin-si (KR); Meehyein Kim, Yongin-si (KR); Doo-Hong Park, Yongin-si (KR); Yeup Yoon, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: MOGAM BIOTECHNOLOGY RESEARCH INSTITUTE, Yongin-si, Gyeonggi-do (KR)

(21) Appl. No.: 12/678,469

(22) PCT Filed: May 8, 2008
(86) PCT No.: PCT/KR08/02589

§ 371 Date: Mar. 16, 2010

Related U.S. Application Data
(60) Provisional application No. 60/972,914, filed on Sep. 17, 2007.

(51) Int. Cl. A61K 31/713; C07H 21/02; A61K 9/127; A61P 31/20

(52) U.S. Cl. 424/450; 536/24.5; 514/44 A

(57) ABSTRACT

A method for enhancing the serum stability and lowering the immunostimulatory property of a small interfering ribonucleic acid (siRNA) which mediates RNA interference (RNAi) against a viral gene expression of hepatitis B virus (HBV) or hepatitis C virus (HCV) is provided.

### FIELD OF THE INVENTION

The present invention relates to a method for enhancing the serum stability and lowering the immunostimulatory property of a small interfering ribonucleic acid (siRNA) which mediates RNA interference (RNAi) against a viral gene expression of hepatitis B virus (HBV) or hepatitis C virus (HCV), which comprises modifying a selected residue of the siRNA.

### BACKGROUND OF THE INVENTION

Small interfering RNAs (siRNAs), sometimes known as short interfering RNAs, are a class of about 19-29 nucleotide-long double-stranded RNAs (dsRNAs) that play a variety of roles in biology. Most notably, an siRNA is involved in the RNA interference (RNAi) pathway where the siRNA interferes with the expression of a specific gene by destroying messenger RNAs (mRNAs) that share sequence homology with the siRNA.

RNAi is a post-transcriptional gene regulation system that is conserved throughout many eukaryotic organisms and recently it has emerged to be a very powerful alternative to the previous technologies to silence gene expression at the mRNA level.

An siRNA is apparently recycled much like a multiple-turnover enzyme, one siRNA molecule being capable of destroying approximately 1,000 mRNA molecules. Therefore, siRNA-mediated RNAi degradation of mRNAs is more effective than any of the currently available technologies for inhibiting the expression of a target gene.

The therapeutic potential of siRNA-induced RNAi degradation has been demonstrated in several recent in vitro studies which include the siRNA-directed inhibition of HIV-1 infection (Novina et al., Nat. Med., 8: 681-686 (2002)) and the suppressed neurotoxic polyglutamine disease protein expression (Xia et al., Nat. Biotech., 20: 1006-1010 (2002)).

siRNAs can also be exogenously (artificially) introduced into cells by various transfection methods to induce knockdown of specific genes. Essentially any gene whose sequence is known can thus be targeted with an appropriate siRNA tailored based on the sequence complementarity. This has made siRNAs an important tool for gene function and drug target validation studies in the post-genomic era.

Many recent studies have focused on improving the specificity and safety of RNAi, for clinical applications (1) by developing systemic siRNA delivery technologies that selectively and efficiently enhance cellular uptake of siRNAs, and (2) by introducing chemical modifications into the siRNA to improve both its serum stability and immuno-safety (Davidson, Nat. Biotechnol., 24:951-952 (2006); Sioud and Furset, J. Biomed. Biotechnol., 2006:23429 (2006)).

There are several examples in the art describing sugar, base and phosphate modifications that can be introduced into nucleic acid molecules for significant enhancement of the nuclease stability and efficacy. For example, oligonucleotides are modified to enhance their stability and/or biological activity with nuclease resistant groups, for example, T-amino, 2′-C-allyl, 2′-flouro and 2′-O-methyl. Sugar modification of nucleic acid molecules have been extensively described in the art (see PCT International Publication Nos. WO 92/07065, WO 93/15187, WO 97/26270 and WO 98/13526; all these publications are hereby incorporated in their totality by reference herein).

An RNA, modified by adding protection groups to the nucleotides or by changing the backbone of the polynucleotide chain, may gain improved stability. However, several forms of modified RNA molecules that are more resistant to RNase degradation than natural RNA have reduced RNAi capability (Parrish et al., Mol. Cell., 5:1077-87 (2000)).

Further, several studies have shown that an siRNA encapsulated in a cationic delivery vehicle can stimulate the innate immune response by activating Toll-like receptors (TLRs) such as TRL3, TLR7 and TLR8 (Hornung et al., Nat. Med., 11:263-270 (2005); Iwasaki and Medzhitov, Nat. Immunol., 5:987-995 (2004); Judge et al., Nat. Biotechnol., 23:457-462 (2005)) or cytoplasmic RNA-binding proteins such as dsRNA-dependent protein kinase (PKR) and retinoic acid inducible gene-1 (RIG-1) (Marques et al., Nat. Biotechnol., 24:559-565 (2006); Sledz et al., Nat. Cell. Biol., 5:834-839 (2003)). It has also been shown that certain RNA sequence motifs, or a high GU- or U-content in the siRNA molecule, are important determinants in stimulating the expression of inflammatory cytokines and interferons (IFNs), and that chemical modification of such moieties abrogates undesirable immune responses in human peripheral blood mononuclear cells (PBMC) and in mice (Hornung et al., supra; Judge et al., supra; Sioud, Eur. J. Immunol. 36:1222-1230 (2006)).

Therefore, there are a need to develop a more robust method of introducing into cells siRNA molecules having increased stability and reduced immunotoxicity while maintaining its RNAi capability.

### SUMMARY OF THE INVENTION

Through extensive research and development efforts, therefore, the present inventors have successfully developed a method of introducing into cells siRNA molecules with increased stability and the reduction of unintended immune response associated with unmodified siRNA while maintaining its RNAi capability.

Accordingly, it is an object of the present invention to provide a method for enhancing serum stability and lowering immunostimulatory property of an siRNA, which is a RNA duplex consisting of a sense strand and an antisense strand and mediating RNAi against a viral gene expression of HBV or HCV, by modifying only uridine residue in the sense strand of the siRNA without modifying any residue in the antisense strand of the siRNA.

Further, the present invention is directed to an siRNA having a pair of nucleotide sequences as set forth in SEQ ID NOS: 1 and 2, a pair of nucleotide sequences as set forth in SEQ ID NOS: 3 and 4 or a pair of nucleotide sequences as set forth in SEQ ID NOS: 5 and 6, whose uridine residue of the sense strand of each siRNA is modified by converting the T-OH group of its ribose ring with a 2′-O-methyl group or 2′-fluoro group, to increase the serum stability and lower the innate immune response of the siRNA while maintaining its RNAi capability for HBV or HCV.

Thus, the present invention provides a method of treating Hepatitis B or Hepatitis C disease in a subject comprising: administering to a subject an effective amount of said modified siRNA.

### BRIEF DESCRIPTION OF THE DRAWINGS

The above and other objects and features of the present invention will become apparent from the following description of the invention, when taken in conjunction with the accompanying drawings, in which:

FIGS. 1A and 1B show serum stability of unmodified and chemically-modified siRNAs;

FIG. 2A represents IFN stimulation of the innate immune response by DTC-Apo-encapsulated unmodified siRNA in vivo;

FIG. 2B illustrates cytokine stimulation of the innate immune response by DTC-Apo-encapsulated unmodified siRNA in vivo;

FIG. 3A depicts inhibition of IFN induction by chemically-modified siHBx1 molecules encapsulated in DTC-Apo liposomes compared to unmodified siHBx1 in vivo;

FIG. 3B describes inhibition of IFN induction by chemically-modified siHBx3 and siHCV molecules encapsulated in DTC-Apo liposomes compared to unmodified siHBx3 and siHCV in vivo, respectively;

FIG. 4A presents a graph of in vivo gene silencing activity of unmodified and chemically-modified siHBx1 duplexes encapsulated in DTC-Apo liposomes in a mouse model of HBV;

FIG. 4B offers northern blot analysis of the gene silencing activity of unmodified siHBx1 and siHBx-OMe-U.

FIG. 5A shows a dose-dependent reduction of core protein expression by DTC-Apo-encapsulated unmodified siHCV in vivo.

FIGS. 5B and 5C show an improved gene silencing activity of siHCV-OMe-U encapsulated within DTC-Apo by using Western blot (FIG. 5B) and RT-PCR analysis (FIG. 5C) from liver tissues.

### DETAILED DESCRIPTION OF THE INVENTION

The present invention provides a method for enhancing serum stability and lowering immunostimulatory property of an siRNA, which is a RNA duplex consisting of a sense strand and an antisense strand and mediating RNAi against a viral gene expression of HBV or HCV, by modifying only uridine residue in the sense strand of the siRNA without modifying any residue in the antisense strand of the siRNA.

In the present invention, any siRNA may be employed if it mediates RNA interference (RNAi) against a viral gene expression of HBV or HCV.

The sense and antisense strands of the present siRNA can comprise two complementary, single-stranded RNA molecules or can comprise a single molecule in which two complementary portions are base-paired. One or both strands of the siRNA of the invention can also comprise a 3′ overhang. As used herein, a “3′ overhang” refers to at least one unpaired nucleotide extending from the 3′-end of a duplexed RNA strand.

In an embodiment in which both strands of the siRNA molecule comprise a 3′ overhang, the length of the overhangs can be the same or different for each strand. In a preferred embodiment, the 3′ overhang is present on both strands of the siRNA, and is 2 nucleotides in length. For example, each strand of the siRNA of the invention can comprise 3′ overhangs of dithymidylic acid (“TT”) or diuridylic acid (“uu”).

In the present method, the siRNA has 19 to 29 nucleotides in length, preferably, 21 to 27 nucleotides in length, and more preferably, 21 or 27 nucleotides in length.

The siRNA of the invention can be obtained using a number of techniques known to those of skill in the art. For example, the siRNA can be chemically synthesized or recombinantly produced using methods known in the art. Preferably, the siRNA of the invention are chemically synthesized using appropriately protected ribonucleotide phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

In accordance with a preferred embodiment of the present invention, the siRNA having a pair of nucleotide sequences as set forth in SEQ ID NOS: 1 and 2 (referred to as “siHBx1”), or a pair of nucleotide sequences as set forth in SEQ ID NOS: 3 and 4 (referred to as “siHBx3”) may be used as a HBV-specific siRNA. Also the siRNA having a pair of nucleotide sequences as set forth in SEQ ID NOS: 5 and 6 (referred to as “siHCV”) may be used as a HCV-specific siRNA.

The nucleotide sequence of the sense strand of the siHBx1, siHBx3 and siHCV are described in Table 1 below. Each strand of the siRNAs of the present invention has 3′ overhangs of dithymidylic acid (“TT”) to form 21 nucleotides in length.

TABLE 1

| | Nucleotide sequence of | |
|---|---|---|
| siRNAs | sense strand | SEQ ID NO. |
| siHBx1 | 5′-GAGGACUCUUGGACUCUCA-3′ | SEQ ID NO: 1 |
| siHBx3 | 5′-CGUCCGACCUUGAGGCAUA-3′ | SEQ ID NO: 3 |
| siHCV | 5′-GGCGACAGCCUAUCCCCAA-3′ | SEQ ID NO: 5 |
| siCont | 5′-ACUACCGUUGUUAUAGGUG-3′ | SEQ ID NO: 7 |

*siCont means a non-specific control siRNA

siHBx1 and siHBx3 targets nucleotides 1653-1673 and 1682-1702, respectively, in the X coding region of the HBV genome (Shin et al., Virus Res., 119:146-153 (2006)). The siHCV targets nucleotides 521-541 in the core coding region of the HCV genome (Kim et al., Virus Res., 122:1-10 (2006)).

The modification of the sense strand and/or antisense strand of the siRNA may be carried out by a known method in the art.

In accordance with the present invention, the sense strand and/or antisense strand of siHBx1, siHBx3 and siHCV are chemically modified with 2′-O-methyl (2′-OMe) or 2′-fluoro (2′-F) at 2′-OH position of its ribose ring. Specifically, the uridine (U) residue in the sense strand of the siRNAs is modified by converting the 2′-OH group of its ribose ring with 2′-OMe group.

Various chemically-modified siRNAs used in the present invention are listed in Table 2 below.

TABLE 2

| siRNA | Chemical modification | |
|---|---|---|
| identification | Sense strand | Antisense strand |
| unmodified | unmodified | unmodified |
| OMe-U | U residue is modified with | unmodified |
| | 2′-OMe | |
| OMe-UC | U and C residues are | unmodified |
| | modified with 2′-OMe | |
| OMe-UG | U and G residues are | unmodified |
| | modified with 2′-OMe | |
| OMe-UA | U and A residues are | unmodified |
| | modified with 2′-OMe | |
| F-UC | U and C residues are | unmodified |
| | modified with 2′-F | |
| OMe/OMe-UC/UC | U and C residues are | U and C residues are |
| | modified with 2′-OMe | modified with 2′-OMe |
| OMe/F-UC/UC | U and C residues are | U and C residues are |
| | modified with 2′-OMe | modified with 2′-F |

For example, “siHBx1-OMe-U” means an siRNA in which uridine (U) residue in the sense strand of siHBx1 is modified with 2′-OMe and no residue in antisense strand of siHBx 1 is modified. Further, “siHBx1-OMe/OMe-UC/UC” means an siRNA in which pyrimidine residues, i.e., uridine (U) and cytidine (C), in the sense strand are modified with 2′-OMe and U and C in the antisense strand are also modified with 2′-OMe.

Further, the present invention provides an siRNA having a pair of nucleotide sequences as set forth in SEQ ID NOS: 1 and 2, a pair of nucleotide sequences as set forth in SEQ ID NOS: 3 and 4 or a pair of nucleotide sequences as set forth in SEQ ID NOS: 5 and 6, whose uridine residue of the sense strand of each siRNA is modified by converting the 2′-OH group of its ribose ring with a T-OMe group or T-fluoro group, to increase the serum stability and lower the innate immune response of the siRNA while maintaining its RNAi capability for HBV or HCV.

Furthermore, the present invention provides a method of treating Hepatitis B or Hepatitis C disease in a subject comprising: administering to a subject an effective amount of said modified siRNA. The subject may be a mammal including a human.

As used herein, an “effective amount” of the siRNA is an amount sufficient to cause RNAi-mediated degradation of the HBV or HCV mRNA in a subject. One skilled in the art can readily determine an effective amount of the siRNA of the invention to be administered to a given subject, by taking into account factors such as the body size and body weight of the subject; the age, health and sex of the subject; the route of administration; and whether the administration is local or systemic. Generally, an effective amount of the siRNA of the invention ranges from about 1 nanomolar (nM) to about 100 nM, preferably from about 2 nM to about 50 nM, more preferably from about 2.5 nM to about 10 nM. It is contemplated that greater or lesser amounts of siRNA can be administered.

In the present method, the present siRNA can be administered to the subject in conjunction with a delivery reagent. Suitable delivery reagents for administration in conjunction with the present siRNA include, but not limited to, liposome, polymer, a mixture of liposome and protein and a mixture of polymer and protein. Liposomes may be lipofectin or lipofectamine. In a preferred embodiment, apolipoprotein A-I-decorated DTC liposome (DTC-Apo) specifically targeting a liver may be used as a preferred delivery reagent. DTC-Apo may be prepared by incubating DTC, which may be obtained by mixing DOTAP (1,2-dioleoyl-3-trimethylammonium-propane) and cholesterol, with apolipoprotein A-I (apo A-I; GenBank Accession No. NM_{—}000039) at a lipid/protein ratio of 10:1 (w/w) overnight at 4° C. Apo A-I may be obtained by isolating and purifying it from human plasma or employing a recombinant vector producing it. DTC-Apo effectively delivers siRNAs to liver cells or tissues for the purpose of RNA interference with high efficiency and low toxicity.

RNAi-mediated degradation of the target mRNA can be detected by measuring levels of the target mRNA or protein in the cells of a subject, using standard techniques for isolating and quantifying mRNA or protein. For example, siRNA of the invention can be delivered to cultured cells, and the levels of target mRNA can be measured by Northern blot technique or by quantitative RT-PCR.

In a preferred embodiment, the serum stability of the chemically-modified siRNAs was measured. Modification of ribonucleic acids at their pyrimidine positions can dramatically enhance serum stability. Interestingly, the stability of siHBx1-OMe-U, in which 2′-OMe substitutions are restricted to sense-strand U residues, was similar to siRNAs with double chemical modifications, such as siHBx1-OMe-UC and siHBx1-OMe-UA. These data suggest that the serum stability of chemically-modified siRNA duplexes is determined by the composition and/or position of modified nucleotide sequences as well as the number.

One of specific embodiments regarding the immunostimulatory and RNAi activity of the present chemically-modified siRNAs reveals that unmodified siRNAs, e.g., siHBx1, siHBx3 and siHCV, encapsulated in DTC-Apo liposomes, activated IFN and inflammatory cytokine responses, which suggests that the targeting moiety has no effect on the uptake of liposome/siRNA complexes by innate immune responses or the stimulation of TLRs by siRNAs.

Consistent to some previous reports (see e.g., Chiu and Rana, RNA, 9:1034-1048 (2003)), but contradictory to others (see e.g., Morrissey et al., Nat. Biotechnol., 23:1002-1007 (2005); Morrissey et al., Hepatology, 41:1349-1356 (2005)), global chemical modification of the 2′-ribose position of all pyrimidines with either 2′-OMe or 2′-F on both strands resulted in a severe reduction in silencing activity, although siRNAs modified in this manner had increased serum stability and did not activate innate immune response pathways. However, chemical modification of either U alone or U and A residues of the siRNA sense strand with 2′-OMe efficiently reduced innate immune activity, and had a more potent effect on the inhibition of viral antigen expression than unmodified siRNA.

Such results suggest that need for chemical modification of therapeutic siRNAs on both strands should be determined based on the primary sequence. The present inventors demonstrated that 2′-OMe modification of U or UA residues of the sense strand of siHBx1 was sufficient to enhance their anti-HBV properties, as they maintained high levels of RNAi activity and immunosafety, and also possessed increased resistance to serum nuclease degradation. Interestingly, with respect to immunostimulation, 2′-OMe modification of siHBx1 at U and C residues still induced both type I and II IFN after systemic administration.

Further, the present inventors also showed that 2′-modification of U residues of the sense strand alone is sufficient to eliminate the global immune response to siRNA. This indicates that the use of UC-modification, which is a generally accepted method for improving the nuclease-resistance of synthetic RNAs, should be evaluated when screening for non-immunostimulatory siRNAs.

The following Examples are intended to further illustrate the present invention without limiting its scope. The animal studies were conducted in accordance with the Guidelines for the Care and Use of Laboratory Animals prepared by the National Academy of Sciences and were approved by Mogam's institutional animal care committee.

### Example 1

### Preparation of Chemically-Modified siRNAs

As defined in Tables 1 and 2 above, various chemically-modified siRNAs were prepared. Specifically, all the siRNAs used in the present invention except 2′-F-modified siRNA (siHBx1-F-UC) were chemically synthesized by Bioneer Co. (Daej eon, South Korea) and siHBx-F-UC was purchased from Dharmacon (Lafayette, Colo.). They were received as pre-annealed duplexes and analyzed by nondenaturing polyacrylamide gel electrophoresis (PAGE).

### Example 2

### Measurement of the Serum Stability of Chemically-Modified siRNAs

In order to investigate the serum stability of unmodified and chemically-modified siRNAs as listed in Tables 1 and 2 above, were dissolved in RNase-free water containing 10% human serum (Sigma) at a final concentration of 10 siRNA. Aliquots were incubated at 37° C. for 0, 1, 3, 6, 24 and 48 hours, and immediately stored at −72° C. siRNAs were separated by 15% nondenaturing PAGE and visualized by ethidium bromide (EtBr) staining.

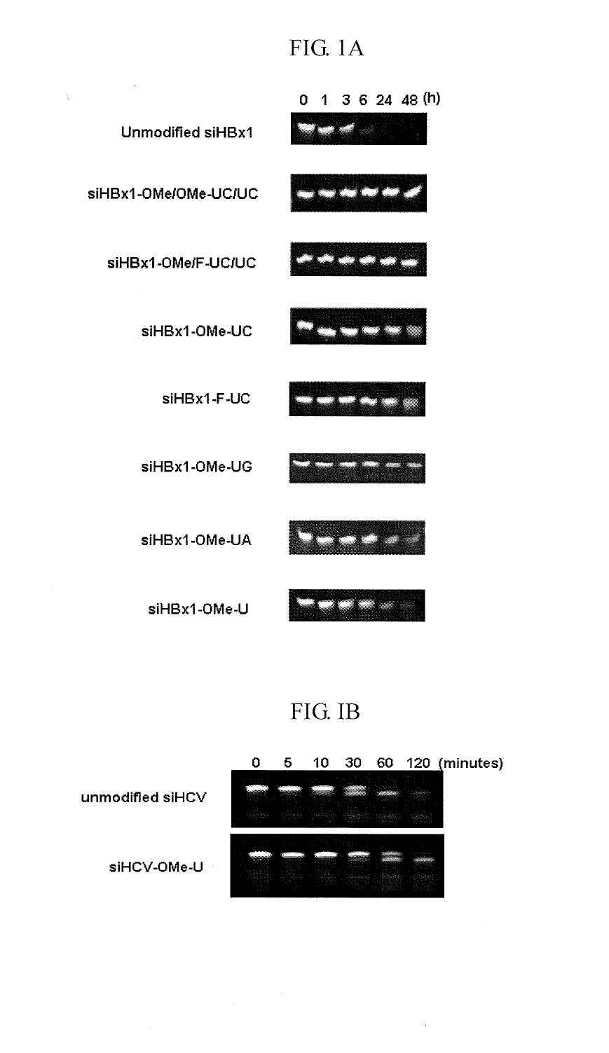

As shown in FIG. 1A, unmodified siHBx1 was degraded nearly to completion after 6 hours incubation with 10% human serum (t_{1/2}=3.6 hours). In contrast, modified derivatives of siHBx1, in particular, siHBx1-OMe/OMe-UC/UC and siHBx1-OMe/F-UC/UC, in which the sense strand pyrimidine residues (U and C) were modified with 2′-OMe and the antisense strand U and C were modified with 2′-OMe and 2′-F, respectively, remained intact over a period of 48 hours. This result clearly shows that modification of ribonucleic acids at their pyrimidine positions can dramatically enhance serum stability. In a series of selective modification, siHBx1-OMe-UC, siHBx1-OMe-UG and siHBx1-OMe-UA, which contains 2′-OMe substitution at the indicated sequences of the sense strand, had half-lives in human serum of 14.0, 45.2 and 10.2 hours, respectively. Among those three siRNAs, siHBx1-OMe-UG had the most improved serum RNase-resistance in vitro. Interestingly, the stability of siHBx1-OMe-U (t_{1/2}=11.3 hours), in which 2′-OMe substitution was restricted to sense-strand U residues, was similar to siRNAs with double chemical modifications, such as siHBx1-OMe-UC and siHBx1-OMe-UA. These data suggest that the serum stability of chemically-modified siRNAs is determined by the composition and/or position of modified nucleotide sequences as well as the number.

Further, as shown in FIG. 1B, chemically-modified siHCV, i.e., siHCV-OMe-U, showed a slightly higher nuclease resistance compared with unmodified siHCV.

### Example 3

### Encapsulation of siRNAs

As taught in a known method in the art, conventional cationic liposomes (DTC) were prepared by mixing DOTAP (Avanti Polar Lipids, Alabaster, Ala.) and cholesterol (Sigma, St. Louis, Mo.) in an equimolar ratio in chloroform (Sigma) (Kim et al., Cancer Res., 63:6458-6462 (2003); Templeton et al., Nat. Biotechnol. 15:647-652 (1997)). After mixing, chloroform was evaporated under a stream of N₂ gas and a lipid film was placed in a vacuum desiccator for 2 hours. The resulting dried lipid film was hydrated in a 5% dextrose solution, followed by sonication using a bath sonicator. In order to prepare apo A-I-decorated DTC liposomes (DTC-Apo), DTC were incubated with human plasma-derived apo A-I at a lipid/protein ratio of 10:1 (w/w) overnight at 4° C. For in vivo administration of the synthetic siRNAs, 40 μg of each siRNA listed in Tables 1 and 2, which were prepared in Example 1, was added to 400 μg of DTC-Apo liposomes in 5% dextrose, and then incubated at room temperature for 30 minutes immediately before use.

### Example 4

### Measurement of the Immune Response of Unmodified and Chemically-Modified siRNAs

Female C57BL/6 mice were purchased from Charles River Laboratories (Wilmington, Mass.). All mice were 7-8 weeks of age and approximately 18-20 g. The mice were divided into four (4) groups (3 mice/each group) and each group of mice was injected intravenously with naked (unmodified) siRNA, empty DTC-Apo liposomes, DTC-Apo/unmodified siRNA complexes, and DTC-Apo/poly(I:C) complexes, respectively, at a dose of 2 mg/kg (about 40 μg/mouse) of siRNA. Poly(I:C) stands for polyinosinic:polycytidylic acid. Poly(I:C) is an immunostimulant and is used to simulate viral infections (Fortier et al., Am. J. Physiol. Regul. Integr. Comp. Physiol., 287:759-66 (2004)). DTC-Apo/poly(I:C) complexes were used as an internal control for monitoring the typical immune response to foreign dsRNA molecules.

In order to investigate the effect of DTC-Apo particles on the immune response when injected with chemically-modified siRNAs into animals by intravenous administration, unmodified siHBx1 formulated with DTC-Apo particles was administered intravenously to mice. To determine their in vivo potency, we determined the induction of IFNs and inflammatory cytokines six (6) hours after intravenous administration.

Serum cytokine levels were determined by measuring mouse IFN-α, IFN-γ (Pierce, Rockford, Ill.), IL-6 and TNF-α (BD Biosciences, San Diego, Calif.) using sandwich ELISA kits, according to the manufacturer's instructions. Two or three independent experiments were performed, and samples were measured in triplicate.

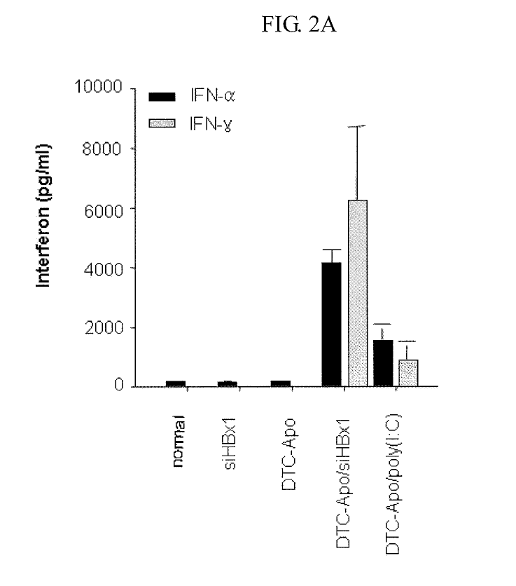

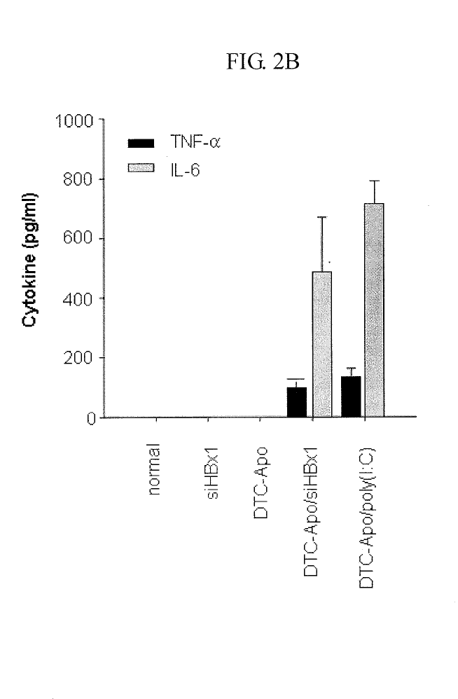

As can be seen from FIGS. 2A and 2B, injection of unmodified (naked) siRNA or DTC-Apo liposomes alone did not activate innate immunity, consistent with previous reports (Ma et al., Biochem. Biophys. Res. Commun., 330:755-759 (2005); Morrissey et al., Nat. Biotechnol., 23:1002-1007 (2005)). In contrast, there was a remarkable increase in serum interferon and cytokine levels in DTC-Apo/unmodified siHBx1-treated animals.

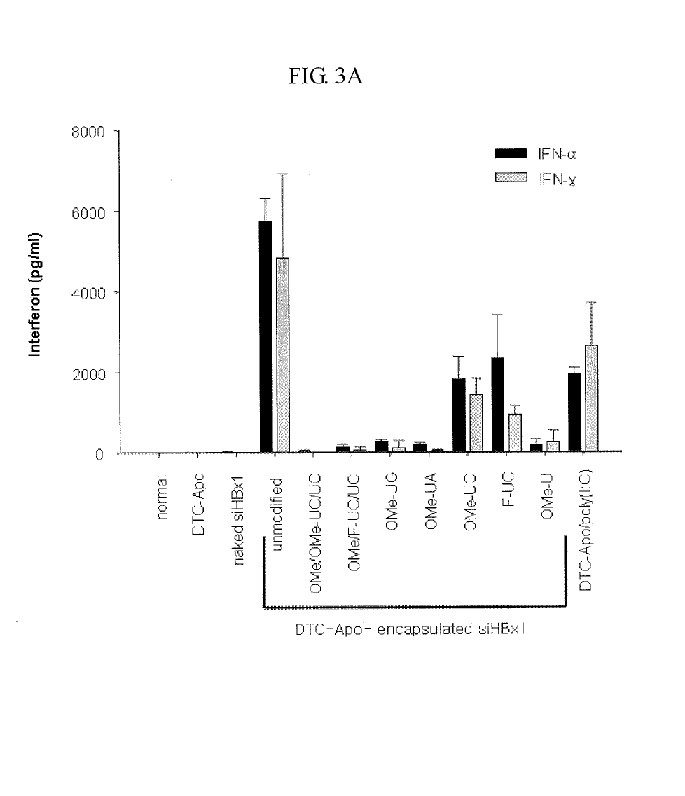

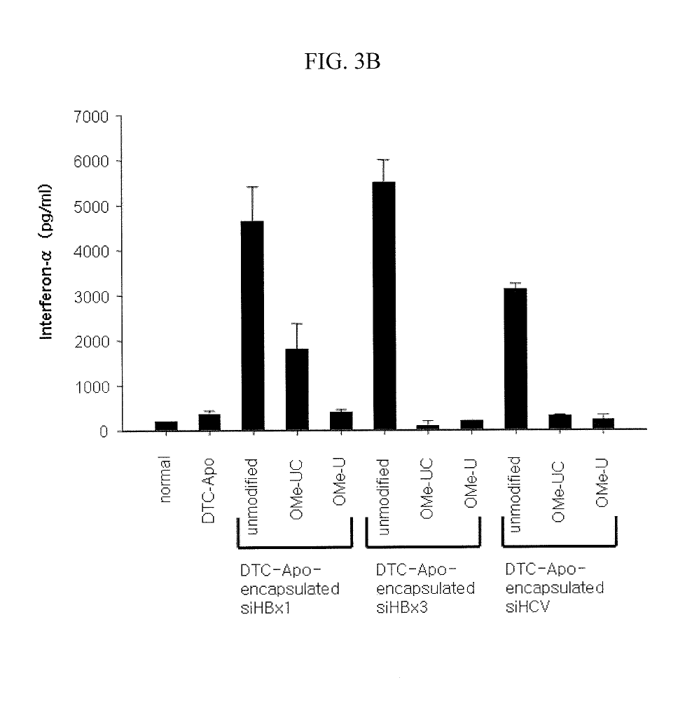

Next, we studied the immunostimulatory properties of chemically-modified siRNAs encapsulated in DTC-Apo particles. DTC-Apo particles containing unmodified (naked) siRNA and chemically-modified siRNAs, DTC-Apo particles, or unmodified siRNA alone were intravenously injected into mice, and six (6) hours later, type I and II IFN levels were measured (FIG. 3A). In contrast to unmodified siHBx1, injection of siHBx1 with chemical modification of pyrimidine residues of both RNA strands (siHBx1-OMe/OMe-UC/UC and siHBx1-OMe/F-UC/UC) formulated with DTC-Apo reduced both serum IFN-α and -γ to nearly normal levels. Activation of IFN was efficiently abrogated by double-modification at UG or UA sequences of the sense strand (siHBx1-OMe-UG and siHBx1-OMe-UA), but not UC sequences (siHBx1-OMe-UC). This immunostimulatory property of 2′-ribose modifications at pyrimidine sequences was confirmed by the use of 2′-F-substituted siHBx1, siHBx1-F-UC. Particularly, injection of an siRNA with minimal modification of sense-strand U residues (siHBx1-OMe-U) circumvented the cationic lipid/siRNA-mediated immune response.

To determine whether the immunostimulatory properties of 2′-ribose modification of pyrimidines was dependent on the primary sequence of the siRNA, we prepared additional synthetic siRNAs that targeted a second HBV X site (siHBx3) or the HCV core region (siHCV) with or without 2′-OMe modification of U alone, or pyrimidines (U and C) of the sense strand. As can be seen in FIG. 3B, all modified siHBx3, i.e., siHBx3-OMe-U and siHBx3-OMe-UC, and siHCV, i.e., siHCV-OMe-UC and siHCV-OMe-U, reduced IFN responses to normal levels, indicating that insufficient abrogation of the innate immune response by 2′-OH substitutions of pyrimidine nucleotides is sequence-dependent. This results also show that 2′-modification of U residues of the sense strand alone is sufficient to eliminate the global immune response to siRNA. They indicate that the use of UC-modification, which is a generally accepted method for improving the nuclease-resistance of synthetic RNAs, should be evaluated when screening for non-immunostimulatory siRNAs.

### Example 5

### Measurement of the RNAi Activity of siRNAs

(5-1) RNAi Activity of siHBx1 and siHBx3

In order to examine the in vivo RNAi activity of chemically-modified siHBx1 and siHBx3, C57BL/6 mice were first injected with a replication-competent HBV and then treated with siHBx1 and siHBx3 encapsulated in DTC-Apo liposomes at a dose of 2 mg/kg siRNA.

Groups of four C57BL/6 mice were hydrodynamically injected with 10 μg of a replication-competent HBV vector, pHBV-MBRI, as described previously (Shin et al., Virus Res., 119: 146-153 (2006)). Eight hours after injection, control siRNA (siCont), unmodified siHBx1 and chemically-modified siHBx1 (40 μg/mouse), which are encapsulated into DTC-Apo liposomes, was systemically administered by tail vein intravenous injection. The levels of secreted viral antigen (HBsAg) in serum were quantified by employing a commercial HBsAg ELISA kit (DiaSorin, Stillwater, Okla.) on days 2 and 4 after siRNA treatment. Serum from normal mice was used as the background.

On day 2 after intravenous siRNA treatment, total RNA was extracted from mouse liver lysates using TRIzol reagent (Invitrogen, Carlsbad, Calif.). RNA (50 μg) was separated on 1% agarose-formaldehyde gel, and transferred to Hybond-N+ nylon membrane (Amersham, Piscataway, N.J.). The probe was obtained by amplifying a partial HBx gene sequence in the presence of [α-³²P] dCTP (NEN-PerkinElmer, Waltham, Mass.) using a pair of primers represented by SEQ ID NO: 9 (forward primer) and SEQ ID NO: 10 (reverse primer).

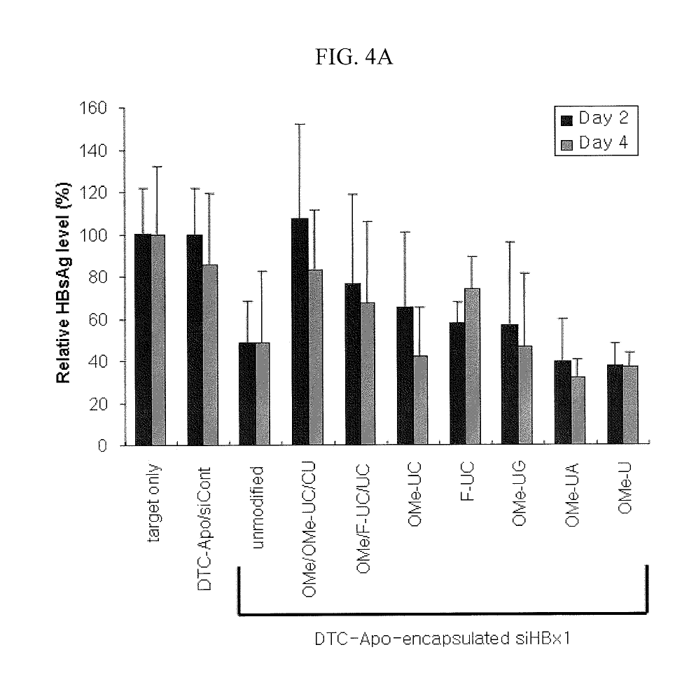

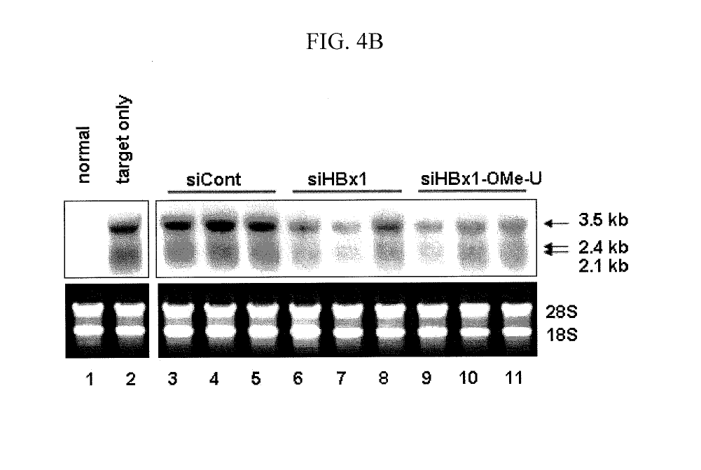

As shown in FIG. 4A, in a group of mice treated with unmodified siHBx1 encapsulated in DTC-Apo liposomes, serum HBsAg decreased by 51.1% (P<0.05) on day 2 and by 43.6% on day 4, relative to the control siRNA-treated group (DTC-Apo/siCont). Further, a group of mice treated with siHBx1-OMe/OMe-UC/UC encapsulated in DTC-Apo liposomes had no detectable ability to block viral protein expression. In contrast to a previous report (Choung et al., Biochem. Biophys. Res. Commun., 342:919-927 (2006)), a group of mice treated with siHBx1-OMe/F-UC/UC encapsulated in DTC-Apo liposomes possessed modest, but not significant, in vivo gene silencing activity. Some siRNAs that contained 2′-OMe- or 2′-F-modified residues in the sense strand, e.g., siHBx1-OMe-UC, siHBx1-OMe-UG and siHBx1-F-UC, were less effective at inhibiting HBsAg expression compared to unmodified siHBx1. This result means that while the sense strand is not the guide strand and thus is not likely to be involved directly in the recognition/cleavage of target mRNA, modification of this strand can, to some extent, interfere with the efficiency of the RNAi machinery in vivo. Notably, systemic administration of two non-immunostimulatory siRNAs, e.g., siHBx1-OMe-U and siHBx1-OMe-UA, reduced HBsAg levels by 60.3% (P<0.01) and 62.4% (P<0.01) on day 2, and by 62.9% (P<0.05) and 56.8% (P<0.05) on day 4, respectively, relative to the siCont-treated group.

Similar in vivo experiments were carried out with 2′-OMe-modified siHBx3 and siHCV and they confirmed that minimal modification of U residues results in producing consistently active and noninflammatory siRNAs in vivo. However, the activity of their dual-modified siRNAs, carrying 2′-OMe-UC, 2′-OMe-UG and 2′-OMe-UA modified residues, was dependent on their primary sequences (data not shown).

The post-transcriptional gene silencing activity of unmodified siHBx1 and its 2′-OMe-U-modified counterpart was also analyzed by Northern blot analysis (FIG. 4B). Both unmodified siHBx1 and chemically-modified siHBx1-OMe-U at single dose reduced viral RNA transcripts in hepatic tissues by an average of 35.7% and 43.2%, respectively, compared to control siRNA (siCont)-treated animals at day 2 after administration. These results support the conclusion that siRNAs with minimal 2′-ribose modification of U residues of the sense strand not only circumvent the bystander immunostimulatory activity mediated by cationic liposomes but also initiate RNAi machinery as potently as unmodified siRNAs.

(5-2) RNAi Activity of siHCV

In order to examine the in vivo gene silencing activity of chemically-modified siHCV, an HCV mouse model was constructed by hydrodynamic injection of 3 μg of pCEP4-HA-CE1E2.

Plasmid pCEP4-HA-CE1E2 can be prepared according to the disclosure of a document known in the art. Specifically, plasmid pCEP4-CE1E2, which was constructed in the document (Kim et al., Virus Res., 122:1-10 (2006)), was modified to achieve hepatocyte-specific and more prolonged expression in vivo (Miao et al., Hum. Gene Ther., 14:1297-1305 (2003)). Briefly, HCR sequence (nucleotides 60 to 325) (Dang et al., J. Biol. Chem., 270:22577-22585 (1995)) and human AAT promoter sequence (Le et al., Blood, 89:1254-1259 (1997)) were substituted for the CMV promoter in the upstream region of the CE1E2 gene of pCEP4-CE1E2 to produce pCEP4-HA-CE1E2.

Eight hours after injection, control siRNA (siCont), unmodified siHCV and chemically-modified siHCV-OMe-U (40 μg/mouse), which are encapsulated into DTC-Apo liposomes, was intravenously administered. On day 2 after siHCV treatment, mice were sacrificed and liver tissues were homogenized. Levels of target protein and RNA expression were determined by immunoblotting and RT-PCR, respectively.

Total cell lysates (30-50 μg) were separated using 12% SDS-PAGE and transferred onto a PVDF membrane (Immobilon-P; Millipore, Billerica, Mass.). HCV core and E2 proteins, and cellular SR-BI and β-actin (as a loading control) proteins were detected using specific antibodies (Kim et al., Mol. Ther. 15:1145-1152 (2007); Kim et al., Virus Res., 122:1-10 (2006)). DTC-assembled apo A-I protein was identified with a goat anti-human apo A-I antibody (Academy Bio-Medical Co., TX). The band intensities were calculated with ImageJ public domain software from the National Institutes of Health (http://rsb.info.nih.gov/ij/).

Total RNA was isolated using TRIzol reagent (Invitrogen). Northern blot analysis was performed with a ³²P-labeled, HCV core probe amplified with a pair of primers represented by SEQ ID NO: 11 (forward primer) and SEQ ID NO: 12 (reverse primer) as described previously (Shin et al., Virus Res., 119:146-153 (2006). For semiquantitative RT-PCR, RNA (1 μg) was reverse transcribed with random hexamers (Invitrogen) and AMV-RT (Promega, Madison, Wis.). The resulting cDNA was amplified with a pair of HCV E2-specific primers represented by SEQ ID NO: 13 (forward primer) and SEQ ID NO: 14 (reverse primer) and a pair of 13-actin-specific primers represented by SEQ ID NO: 15 (forward primer) and SEQ ID NO: 16 (reverse primer) separately. The PCR products were electrophoresed on a 2% agarose gel.

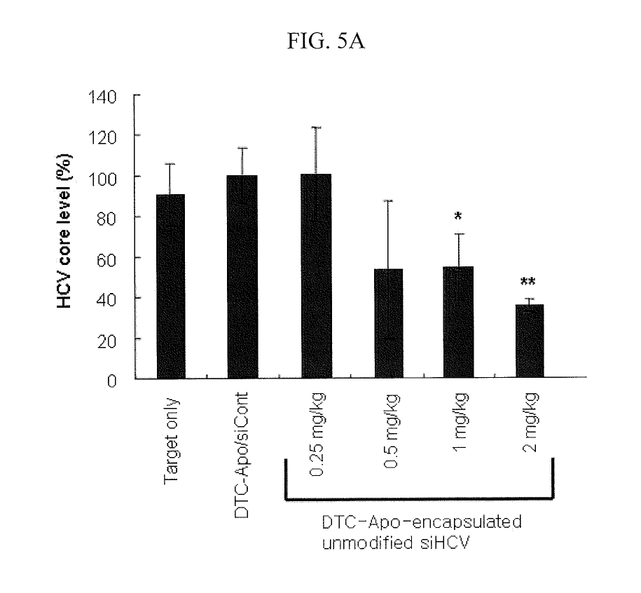

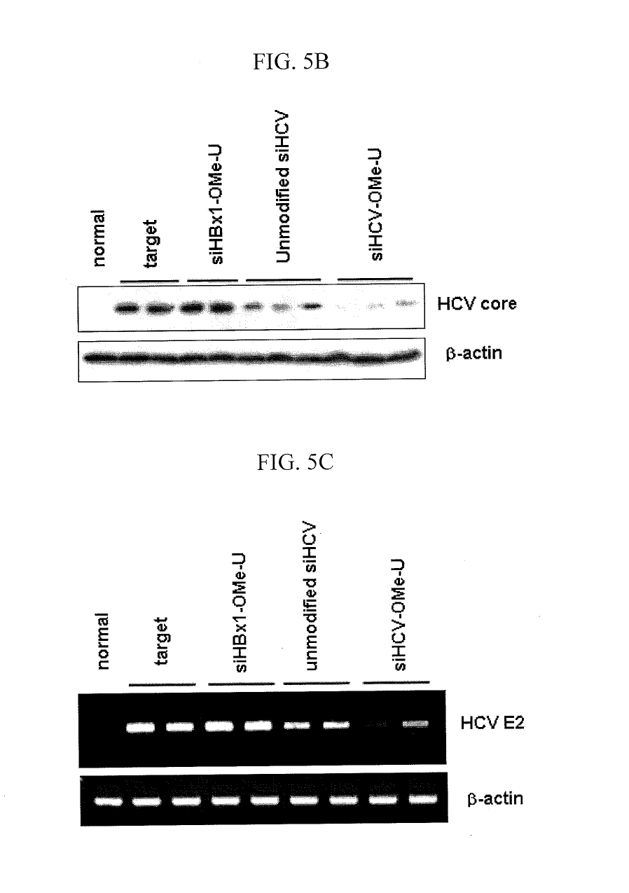

Relative hepatic core protein expression levels were quantified on day 2 after intravenous administration by measuring the corresponding band intensity from western blot analysis of DTC-Apo/siCont (2 mg/kg) and DTC-Apo/unmodified siHCV at an siHCV dose of 0.25, 0.5, 1 and 2 mg/kg (*P<0.05 and **P<0.0005 versus DTC-Apo/siCont-treated group). As shown in FIG. 5A, unmodified siHCV encapsulated in DTC-Apo silenced the target protein in a dose dependent manner and average core protein levels were reduced by 45.5% (P<0.05) or 64.4% (P<0.0005) in mice treated with 1 mg/kg or 2 mg/kg siHCV, respectively.

Next, we tested the gene silencing potency of siHCV-OMe-U in mice (FIGS. 5B and 5C). The gene silencing effect, as evidenced by protein level, was detected in both DTC-Apo/unmodified siHCV and DTC-Apo/siHCV-OMe-U-injected animals. Notably, non-immunostimulatory siHCV-OMe-U reduced viral core protein by 85.5% (P<0.001), whereas chemically-modified HBV siRNA, siHBx1-OMe-U, exhibited no activity (FIG. 5B). We also measured E2 RNA levels using semi-quantitative RT-PCR. The results showed that viral RNA levels, in mice receiving a single dose of unmodified siHCV or siHCV-OMe-U, were reduced by 44.8% or 69.2% (FIG. 5C). These experiments showed that 2′-OMe-modified siHCV is more potent than unmodified siHCV.

Administration of DTC-Apo particles containing chemically-modified siHCV, e.g., siHCV-OMe-U, which is obtained by 2′-OMe modification at two U residues in the sense strand of siHCV, at a single dose of 2 mg siHCV/kg inhibited improved target gene silencing activity (85% knockdown) without immunotoxicity.

While the invention has been described with respect to the above specific embodiments, it should be recognized that various modifications and changes may be made to the invention by those skilled in the art which also fall within the scope of the invention as defined by the appended claims.

What is claimed is:

1. A method for enhancing the serum stability and lowering the immunostimulatory property of a small interfering ribonucleic acid (siRNA), which is a RNA duplex consisting of a sense strand and an antisense strand and mediates RNA interference (RNAi) against the expression of hepatitis B virus (HBV) or hepatitis C virus (HCV), which comprises modifying only the uridine residue in the sense strand of the siRNA without modifying any residue in the antisense strand of the siRNA.

2. The method of claim 1, wherein the uridine residue in the sense strand of the siRNA is modified by converting the 2′-OH group of its ribose ring with a 2′-O-methyl group or T-fluoro group.

3. The method of claim 1, wherein the siRNA has 19 to 29 nucleotides in length.

4. The method of claim 3, wherein the siRNA has 21 to 27 nucleotides in length.

5. The method of claim 3, wherein the siRNA has 21 or 27 nucleotide in length.

6. The method of claim 3, wherein the siRNA has a pair of nucleotide sequences as set forth in SEQ ID NOS: 1 and 2, a pair of nucleotide sequences as set forth in SEQ ID NOS: 3 and 4, or a pair of nucleotide sequences as set forth in SEQ ID NOS: 5 and 6.

7. An siRNA having a pair of nucleotide sequences as set forth in SEQ ID NOS: 1 and 2, a pair of nucleotide sequences as set forth in SEQ ID NOS: 3 and 4, or a pair of nucleotide sequences as set forth in SEQ ID NOS: 5 and 6, whose uridine residue of the sense strand of each siRNA is modified by converting the T-OH group of its ribose ring with a 2′-O-methyl group or 2′-fluoro group.

8. A method of treating Hepatitis B or Hepatitis C disease in a subject comprising: administering to a subject an effective amount of a siRNA of claim 7.

9. The method of claim 8, wherein the siRNA is administered in conjunction with a delivery reagent.

10. The method of claim 9, wherein the delivery agent is selected from the group consisting of liposome, polymer, a mixture of liposome and protein and a mixture of polymer and protein.


SEQUENCE LISTING (16 sequences)

SEQ ID NO: 1 - RNA, 19 nt, Artificial Sequence
  Synthetic construct (sense strand of siHBx1)
gaggacucuuggacucuca

SEQ ID NO: 2 - RNA, 19 nt, Artificial Sequence
  Synthetic construct (antisense strand of siHBx1)
ugagaguccaagaguccuc

SEQ ID NO: 3 - RNA, 19 nt, Artificial Sequence
  Synthetic construct (sense strand of siHBx3)
cguccgaccuugaggcaua

SEQ ID NO: 4 - RNA, 19 nt, Artificial Sequence
  Synthetic construct (antisense strand of siHBx3)
uaugccucaaggucggacg

SEQ ID NO: 5 - RNA, 19 nt, Artificial Sequence
  Synthetic construct (sense strand of siHCV)
ggcgacagccuauccccaa

SEQ ID NO: 6 - RNA, 19 nt, Artificial Sequence
  Synthetic construct (antisense strand of siHCV)
uuggggauaggcugucgcc

SEQ ID NO: 7 - RNA, 19 nt, Artificial Sequence
  Synthetic construct (sense strand of siCont)
acuaccguuguuauaggug

SEQ ID NO: 8 - RNA, 19 nt, Artificial Sequence
  Synthetic construct (antisense strand of siCont)
caccuauaacaacgguagu

SEQ ID NO: 9 - DNA, 20 bp, Artificial Sequence
  Synthetic construct (HBx forward primer)
tgtgctgccaactggatcct

SEQ ID NO: 10 - DNA, 20 bp, Artificial Sequence
  Synthetic construct (HBx reverse primer)
gtaagaccttgggcaagacc

SEQ ID NO: 11 - DNA, 24 bp, Artificial Sequence
  Synthetic construct (forward primer for HCV core probe)
atgagcacaaatcctaaacctcaa

SEQ ID NO: 12 - DNA, 24 bp, Artificial Sequence
  Synthetic construct (reverse primer for HCV core probe)
actaggccgagagccgcggggtga

SEQ ID NO: 13 - DNA, 20 bp, Artificial Sequence
  Synthetic construct (HCV E2-specific forward primer)
cctgcactgtcaactttacc

SEQ ID NO: 14 - DNA, 20 bp, Artificial Sequence
  Synthetic construct (HCV E2-specific reverse primer)
agagcaacaggacatactcc

SEQ ID NO: 15 - DNA, 20 bp, Artificial Sequence
  Synthetic construct (beta-actin-specific forward primer)
gccatgtacgttgctatcca

SEQ ID NO: 16 - DNA, 20 bp, Artificial Sequence
  Synthetic construct (beta-actin-specific reverse primer)
atctcttgctcgaagtccag